(19) United States
(12) Patent Application Publication
(10) Pub. No.: US 2012/0270204 A1
(43) Pub. Date: Oct. 25, 2012

(54) MAGNETIC SORTING OF MAMMALIAN SPERM HAVING DAMAGED MEMBRANES

(76) Inventors: Daniel N. Fox, Fort Collins, CO (US); Tidhar Sadeh, Fort Collins, CO (US); Matthias J.G. Ottenberg, Holmen, WI (US); Kristie M. Melnik, Upper Arlington, OH (US); John L. Schenk, Fort Collins, CO (US)

(21) Appl. No.: 13/398,467

(22) Filed: Feb. 16, 2012

Related U.S. Application Data
(60) Provisional application No. 61/443,365, filed on Feb. 16, 2011.
(60) Provisional application No. 61/576,956, filed on Dec. 16, 2011.

(51) Int. Cl. C12N 13/00; B82Y 25/00; B82Y 5/00

(52) U.S. Cl. 435/2; 977/923; 977/774; 977/773; 977/920

(57) ABSTRACT

A method for magnetic sorting of mammalian sperm cells having damaged membranes is described. In an embodiment of the invention, carboxyl-group functionalized magnetic particles are conjugated to propidium iodide, the resulting composition is mixed with a sample of sperm cells, and sperm cells bound to magnetic particles are separated by magnetic-activated cell sorting.

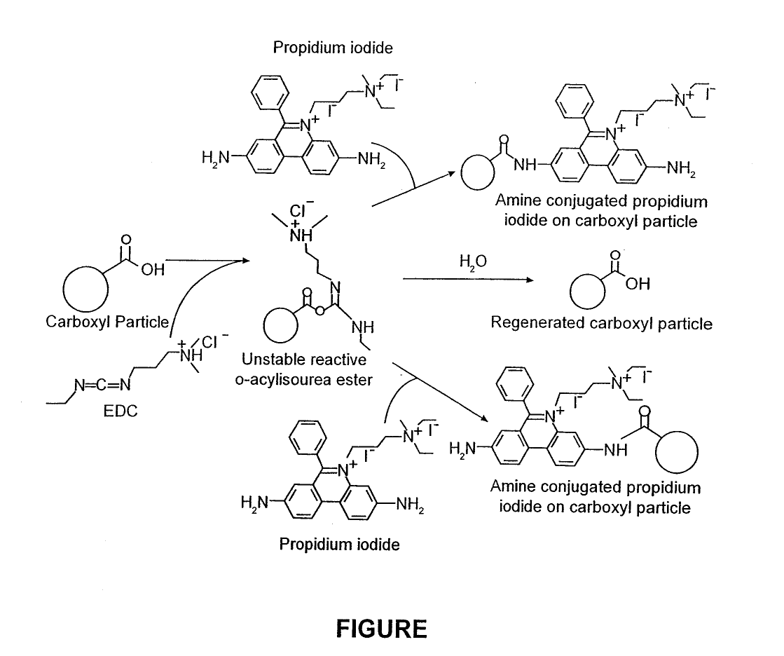

### RELATED CASES

The present application claims the benefit of provisional patent application Ser. No. 61/443,365 for “Magnetic Flow Sorting of Mammalian Sperm Having Damaged Membranes” by John L. Schenk et al., filed on Feb. 16, 2011, and provisional patent application Ser. No. 61/576,956 for “Magnetic Sorting of Mammalian Sperm Having Damaged Membranes” by Daniel N. Fox et al., filed on Dec. 16, 2011, which provisional applications are hereby incorporated by reference herein for all that they disclose and teach.

### BACKGROUND

Increasing the concentration of healthy sperm in a sample improves sperm viability, increases pregnancy rates for both in vitro and in vivo fertilization procedures and improves embryo quality, which are major sources of infertility in mammals.

Early phases of disturbed membrane functions are associated with asymmetry of the membrane phospholipids. For example, the phospholipid phosphatidylserine (PS), which is normally present on the inner leaflet of the plasma membrane, becomes externalized to the outer leaflet, and is a known marker for early stages of apoptosis. Annexin-V has a high affinity for PS, but cannot pass through an intact sperm membrane. However, annexin-V may bind to externalized PS, and has been used for magnetically labeling apoptotic sperm which may then be removed by magnetic separation methods.

### SUMMARY OF THE INVENTION

Embodiments of the present invention overcome the disadvantages and limitations of the prior art by providing a method for removing necrotic sperm cells from a sample of sperm cells.

Another object of embodiments of the present invention is to provide a method for selecting a chosen characteristic of sperm cells.

Additional objects, advantages and novel features of the invention will be set forth in part in the description which follows, and in part will become apparent to those skilled in the art upon examination of the following or may be learned by practice of the invention. The objects and advantages of the invention may be realized and attained by means of the instrumentalities and combinations particularly pointed out in the appended claims.

To achieve the foregoing and other objects, and in accordance with the purposes of the present invention as embodied and broadly described herein, the method for separating sperm cells having damaged membranes from those having intact membranes, hereof, includes: attaching a membrane-impermeable, DNA-binding species to magnetic particles; mixing the resulting magnetic particles with a sample of sperm cells; and separating the sperm cells bound to magnetic particles by magnetic cell sorting.

In another aspect of the present invention, and in accordance with its objects and purposes, the method for selecting sperm cells having a chosen characteristic, hereof, includes: detecting sperm cells having a chosen characteristic; damaging the membranes of sperm cells not having the chosen characteristic, forming thereby a mixture of sperm cells having damaged membranes and sperm having intact membranes; attaching a membrane-impermeable, DNA-binding species to magnetic particles; mixing the resulting magnetic particles with the mixture of sperm cells; and separating the sperm cells bound to magnetic particles by magnetic cell sorting; whereby the unseparated sperm have the chosen characteristic.

Benefits and advantages of the present invention include, but are not limited to, removing sperm cells having varying degrees of membrane damage from a sample of sperm cells.

### BRIEF DESCRIPTION OF THE DRAWINGS

The accompanying drawing, which is incorporated in and forms a part of the specification, illustrates an embodiment of the present invention which, together with the description, serve to explain the principles of the invention. In the drawing:

The FIGURE illustrates the reaction of EDC with a carboxyl group attached to a magnetic particle forming an o-acylisourea intermediate capable of reacting with one of the amine groups on propidium iodide (PI), forming thereby a stable amide bond; the intermediate may also be hydrolyzed to regenerate the original carboxyl group by the addition of H₂O.

### DETAILED DESCRIPTION OF THE INVENTION

Briefly, embodiments of the present invention include a method for removing sperm having damaged membranes from those with intact membranes, thereby enriching sperm viability of a sperm sample. The method can be applied to sperm contained in freshly collected neat ejaculates, after dilution, during and after cooling, or during and after other semen processing procedures that are employed prior to cryopreservation, and to frozen/thawed sperm. The enriched sperm populations can be used for routine artificial insemination, prior to or after sperm sexing techniques, or for in vitro fertilization, for all mammalian sperm.

Additional damage to the membranes of intact sperm is reduced by removing known harmful effects caused by damaged sperm. Specifically, DNA fragmentation, oxidative damage caused by peroxidation, and the premature release of proteolytic and hydrolic enzymes are examples of sperm damage caused by membrane damaged sperm. Damage to spermatozoal integrity reduces sperm lifespan both in vitro and in vivo, reduces fertilization ability, and likely causes poor embryo quality, which is a major source of infertility in mammals.

Sperm pre-capacitation can result in ova fertilization failure. Further, damage to sperm chromatin can result in poor embryo quality. Because fertilization is a time-sensitive event and good embryo quality is essential for timely embryo development, both can be adversely affected by sperm quality. Factors released from damaged sperm may be partly responsible for further cellular damage to the remaining subpopulation of normal sperm. P. Shannon, in “The contribution of seminal plasma, sperm numbers and gas phase to dilution effects of bovine spermatozoa,” J. Dairy Sci., 48: 1357 (1965), reported that freshly killed dead sperm reduced livability of sperm in diluted bovine semen. Further, freshly ejaculated sperm subjected to elevated temperatures before ejaculation were found to exhibit high reactive oxidative species levels. Thus, the toxic effect of dead sperm may be due to their amino acid oxidase activity. Dead and abnormal sperm have toxic (Shannon, P. and Curson, B., “Toxic effect and action of dead sperm on diluted bovine semen,” J. Dairy Sci., 55: 615-620 (1972)) and lytic effects (Lindemann, C. B., Fisher, M. and Lipton, M., “A comparative study of the effects of freezing and frozen storage on intact and demembranated bull spermatozoa,” Cryobiology, 19: 20-28 (1982)) on companion cells in semen, and consequently reduce fertility (Saacke, R. G. and White, J. M., “Semen quality tests and their relationship to fertility,” Proc. 4th National Association of Animal Breeders, Tech. Conf. Artificial Insemination and Reproduction, 18-20 Apr., 1972, Madison, Wis., National Association of Animal Breeders, Columbia, Mo., pp. 22-27).

In embodiments of the present invention, carboxyl group functional magnetic particles ranging in size from 10 nm to 800 nm, and having an average hydrodynamic diameter of 230 nm, were conjugated to lyophilized propidium iodide (PI) by standard EDC/NHS chemistry (EDC is also known as EDAC, EDCI and 1-ethyl-3-(3-dimethylaminopropyl)carbodiimide, and NHS is N-hydroxysuccinimide), where EDC-mediated coupling efficiency increases in the presence of amine reactive esters for the conversion of carboxyl groups to amines. Magnetic particle size may be determined by dynamic light scattering (DLS) analysis and iron concentration through inductive coupled plasma (ICP) analysis. PI is a fluorescent molecule having a molecular mass of 668.4 Da that may be used to stain DNA. PI binds to DNA by intercalating between the bases thereof with little or no sequence preference and with a stoichiometry of one dye per 4-5 base pairs of DNA. PI is membrane impermeant and is generally excluded from viable cells. However, PI may be used to assess sperm viability; that is, whether the plasma membrane is intact.

The current invention differs from prior magnetic sperm separation procedures utilizing annexin-V where only apoptotic sperm are magnetically labeled and are removed during magnetic separation. As stated hereinabove, early phases of disturbed membrane functions are associated with asymmetry of the membrane phospholipids. The phospholipid phosphatidylserine (PS), which is normally present on the inner leaflet of the plasma membrane, becomes externalized to the outer leaflet. The externalization of PS is a known early marker for apoptosis. Annexin-V has a high affinity for PS and, although it cannot pass through an intact sperm membrane, Annexin-V will bind to externalized PS. Embodiments of the present invention can remove necrotic sperm that have been traumatized during sperm processing procedures for sperm cryopreservation. Necrotic sperm damage occurs by different cellular processes than apoptosis, which is a naturally occurring cause of cellular death.

Colloidal super-magnetic microbeads (˜50 nm in diameter) conjugated with annexin-V may be used to separate out apoptotic sperm by magnetic-activated cell sorting. Sperm with PS that has externalized to the outer leaflet will bind to these microbeads. When placed into a column containing iron balls and passed through a strong magnetic field, those cells remain in the separation column. Sperm with intact membranes remain unlabeled and pass freely through the column.

In the present invention, sperm with varying degrees of membrane damage can be labeled with magnetic PI particles. By contrast, annexin-V/microbead magnetic cell sorting procedures fail to bind to disabled or necrotic sperm that do not have externalized PS. Magnetic particles conjugated to PI firmly attach to the DNA of all damaged sperm. When membrane damaged sperm are passed through a magnetic cell separating apparatus, such sperm are eliminated from the general population. The resultant harvested sub-population of normal sperm may be further processed for cryopreservation, sex selection or used in assisted reproductive technologies (ART's).

Embodiments of the present invention can be used with any type of magnetic separating apparatus including, but not limited to, devices incorporating columns, and continuous-throughput proportional magnetic sorting devices, the latter sorting devices having high throughput and consistent and quantitative separation performance without the clogging or reduction in performance associated with column-based devices. By using a high-definition magnetic cell-tracking velocimeter (MCTV) in cooperation with a quadrupole mass spectrometer, the magnetophoretic mobility of the sample may be measured, in the present case, the population of unlabeled sperm compared with the population of labeled sperm, from which the flow rates of the instrument may be adjusted to achieve the desired enrichment of the semen sample.

As will be discussed in more detail hereinbelow, the use of simple magnetic fields applied to containers, for example, a test tube holder having a strong magnetic base, will suffice.

Sperm labeled with PI and subjected to magnetic cell separation can be removed more efficiently and in greater numbers per time unit when compared with flow cytometry. Magnetic cell separation requires a lower internal operating pressure and the stream of fluid containing the sperm does not have to be broken into sperm-damaging droplets as that for flow cytometry. Further, the sheath fluid required for flow cytometry is generally a salt-based, lipo-protein deficient physiological medium. Magnetic cell separation allows sperm to be bathed in nutrient-rich buffers that promote and prolong sperm viability during the separation procedure.

An application for embodiments of the present invention includes removal of dead sperm prior to using a flow cytometer for sex selection. When using normal flow, dead sperm are treated as contaminants; thus, a drop having a dead sperm in it is discarded even if that drop also has a desirable sperm. This necessitates a lower throughput to reduce the number of droplets having both contaminants and desirable sperm. The use of the present invention for removing dead sperm prior to sex selection permits the throughput to be increased, with attendant increased sorted output rates. TABLE 2 shows the results of a simulation varying throughputs and live/dead ratios used to determine the number of sperm per second that could be sorted by counting the simulated desirable sperm in droplets that did not have contaminants, assuming that 80% of the sperm that came through were oriented correctly in the nozzle such that DNA differences could be accurately measured.

TABLE 2

| Live Sperm | Number of Sorts/s |
|---|---|
| 50% | 4,825 |
| 63% | 6,180 |
| 75% | 7,580 |
| 88% | 9,050 |
| 100% | 10,700 |

The results show significant throughput gain when the dead sperm are removed. For example, if a sample of 75% live sperm was increased to be 88% live sperm, the expected sort rate of desirable sperm would increase from 7580 to 9050 per second, a 19.4% increase.

In other magnetic cell separation applications, embodiments of the present invention could be combined with cellular analysis and an intermediate processing step. One such example would be to use standard (non-droplet-forming) flow cytometry to detect a chosen cell type, after which a source of energy, including but not limited to, pulses of laser light or electric charge, is used to selectively kill or render non-functional all but the desired cells. Embodiments of the present invention may then be used to magnetically label and remove the dead or non-functional cells through magnetic cell separation processes. In the example of sex selection of sperm, this procedure would eliminate the need for droplet formation for sorting, thereby eliminating the stresses on sperm caused by shear and impact forces. This leads to higher throughput rates by allowing sperm input rates higher than what otherwise would be used to statistically target only one sperm per droplet.

Having generally described the present method, more details thereof are presented in the following EXAMPLES.

### Example 1

### Particle Preparation for Magnetic Staining of Dead/Damaged Sperm

i. Magnetic Cores:

Magnetic cores were fabricated by coprecipitation of Fe₃O₄ with Fe₂O₃ so that the magnetic susceptibility of the particles in a chosen magnetic field is sufficiently high to provide rapid separation of magnetically labeled cells from nonlabeled cells. The core may be composed of any magnetic material; those most commonly used are: (1) ferrites such as magnetite, zinc ferrite, or manganese ferrite; (2) metals such as iron, nickel or cobalt; and (3) chromium dioxide. In the present example, iron cores are composed principally of magnetite (Fe₃O₄). In other embodiments the cores may include other iron oxide based nanoparticle materials including composites having the general structure MFe₂O₄ (where M may be Co, Ni, Cu, Zn, Mn, Cr, Ti, Ba, Mg, or Pt). In this example a reaction chamber containing 400 ml of dH₂O in a water kettle is prewarmed to 85° C. To the 400 ml of prewarmed dH₂O, 23.4 g of FeCl₃.6H₂O and 8.6 grams of FeCl₂ are added and the mixture is stirred under nitrogen gas. To this solution, 30 ml of 25% NH₃.H₂O is added and mixing continued under nitrogen gas. Almost immediately, the orange salt mixture turns to a dark brown/black solution. The precipitate is collected magnetically and the supernatant is decanted. To the magnetically collected ferrofluid, 800 ml of dH₂O is added, swirled and the magnetic collected process is repeated. This washing process is repeated 4 times to insure that all residual NH₃.H₂O and any nonmagnetic particles are removed. The final wash step includes a solution of 800 ml 0.02 M NaCl in dH₂O. The collected iron core sizes are between approximately 3 nm and approximately 10 nm.

ii. Coating of Iron Cores with a Functionalizable Surface:

The final outer layer consists of a polymer coat that interacts with the aqueous environment and serves as an attachment site for proteins and ligands. Suitable polymers may include polysaccharides, alkylsilanes, biodegradable polymers such as, but not limited to, poly(lactic acids) (PLA), polycaprolactone (PCL), and polyhydroxybutyrate-valerate (PHBV); composites, and polyolefins such as polyethylene in its different variants. More specifically, polysaccharide chains may include dextrans, arabinogalactan, pullulan, cellulose, cellobios, inulin, chitosan, alginates and hyaluronic acid. Alkylsilanes may also be employed to encapsulate the magnetic core. Alkylsilanes suitable for this inventions, include, but are not limited to, n-octyltriethoxysilane, tetradecyltrimethoxysilane, hexadecyltriethoxysilane, hexadecyltrimethoxysilane, hexadecyltriacetoxysilane, methylhexadecyldiacetoxysilane, methyl-hexadecyldimethoxysilane, octadecyltrimethoxysilane, octadecyltrichlorosilane, octadecyltriethoxysilane, and 1,12-bis(trimethoxysilyl)dodecane.

For the examples of magnetic removal of dead/dying sperm described hereinbelow, a silane composition was used to encapsulate the iron cores. The iron core precipitate was allowed to settle, and 10 ml of the settled ferrofluid (˜2 grams of iron) was added to 100 ml of 10% 2-(carbomethoxy)ethyltrimethoxysilane. The pH was adjusted to 4.5 using >99.5% glacial acetic acid, and the suspension was reacted at 90-95° C. for 2 h under nitrogen gas with vigorous mixing. After cooling, the particles were magnetically collected and washed with dH₂O. After the water washing step, the particles were magnetically collected and washed three times with methanol. The particles were again magnetically collected and washed three times with dH₂O. After the third wash, the silane-coated magnetic nanoparticles were resuspended in 5 ml of 0.05 M 2-(N-morpholino)ethanesulfonic acid (MES) Buffer. Iron concentration was adjusted to the quantities required for subsequent EDC activation and propidium iodide coupling by using Inductively Couple Plasma-Optical Electron Spectroscopy (ICP-OES). The particles had an average hydrodynamic diameter of 250 nm, but were found to range from 10 nm to 2 μm.

iii. Coupling of Proteins and Ligands to the Particle Surfaces:

Periodate treatment of dextran and other polymers is a method for the attachment of proteins due largely to the large number of reactive groups that are available for modification. Mild sodium periodate treatment creates reactive aldehyde groups by oxidation of adjacent hydroxyl groups or diols. Proteins, antibodies, streptavidin, and amino-modified nucleic acids may be added at high pH to allow amines to form Schiff bases with the aldehydes. The linkages are subsequently reduced to stable secondary amine linkages by treatment with sodium borohydride or sodium cyanoborohydride, which will reduce unreacted aldehyde groups to alcohols. Another method of coupling proteins to the magnetic nanoparticles is to create stable hydrazine linkages. For example, a protein may be coupled to dextran using succinimidyl 4-hydrazinonicotinate acetone hydrazone (SANH; Solulink Inc, San Diego, Calif.). The reaction uses five-fold less protein, and the resulting protein density appears as high as with other methods. The SANH reagent allows more efficient and gentle coupling of ligands to the dextran surface.

Ligand attachment to silica-coated magnetic nanoparticles may be accomplished using (3-aminopropyl)triethoxysilane (APTS) to introduce amines onto the surface of the particles, while (3-mercaptopropyl)triethoxysilane (MPTMS) is used to introduce SH groups. The heterobifunctional coupling agent (Succinimidyl 4-[N-maleimidomethyl]cyclohexane-1-carboxylate) may then be used to link thiols to the amines. As examples, amines on the particle surface can be linked to thiols on streptavidin molecules, and thiols on the particle surface can be linked to amines on streptavidin. There are several methods of crosslinking proteins through chemical modifications known in the art that can be used for the present embodiments of the invention. As will be described in more detail hereinbelow, proteins and ligands may be attached to the carboxylic acid functionalized silane through EDC chemistry.

iv. EDC Activation of Carboxyl Groups on Particle Surface, and Propidium Iodide Attachment:

The silanized magnetic particles were resuspended in 0.05 M MES buffer, collected magnetically, and the supernatant was aspirated and discarded. Another 5 ml of MES buffer (0.05 M, pH 5.2) per 10 mg of iron was added to the particles and vigorously shaken. Particles were magnetically collected, the supernatant aspirated and discarded. This step was repeated 2 additional times. Frozen EDC was allowed to thaw at room temperature for 30 min. EDC is commonly obtained as a hydrochloride, is a water soluble carbodiimide which is typically employed at pH in the range between 4.0 and 6.0. It can also be used as a chemical crosslinker for collagen, reacting with the carboxylic acid groups of the collagen polymer which can then bond to the amino group in the reaction mixture.

1.6 mg of EDC/mg iron was added to the particle suspension and the suspension was vigorously shaken. Each tube containing particles and EDC was placed on a laboratory rocker at room temperature for 30 min. After 30 min., particles were magnetically collected, the supernatant then being aspirated and discarded. Buffers having various salt concentrations, molarities, including but not limited to, 0.1 M to 1 M, and pH ranges from 10 to 4.7 may be used for protein conjugation to the various surfaces set forth hereinabove. The function of each antibody, protein and ligand optimizes at different pH ranges and molarities, as is known in the art (Hermanson, Bioconjugate Techniques, 2008). The magnetic particles were added to 0.05 M MES buffer, magnetically collected, and the supernatant was aspirated and discarded. This step was repeated three times. 10 mg of propidium iodide was resuspended in 0.05 M MES buffer and added to the particles so that the total labeling volume was 5 ml per 10 mg of iron.

A stoichiometric balance of 1 mg of propidium iodide per 1 mg of iron was used for the coupling reaction since previous experiments indicated that best binding of dead cells occurred at this concentration. The ranges for propidium iodide may include, but are not limited to, 0.125 mg to 5 mg of propidium iodide per mg of iron. Tubes were shaken and placed on a laboratory rocker at room temperature for 24 h, and particles were magnetically collected. The supernatant was aspirated and discarded. Each particle suspension was resuspended in 5 ml of MES buffer. To each tube, 5 ml of quenching solution (1M glycine, pH 8.0) was added and the tubes were vigorously shaken. Quenching solutions may include, but are not limited to, 2-mercaptoethanol, ethanolamine, and glycine. Each tube was then placed on a laboratory rocker for 30 min. at room temperature. After 30 min., 5 ml of tris(hydroxymethyl)aminomethane (TRIS) wash buffer was added to each tube and shaken to mix. The particles were magnetically separated, the supernatant aspirated and discarded. This step was repeated 4 times. After the wash steps, each particle suspension was resuspended in wash buffer so that the resultant working iron concentration was 5 mg/ml as confirmed by ICP-OES. After the conjugation process is complete, particles were magnetically collected, washed and filtered to obtain a size distribution of 50 to 400 nm.

The particles are advantageously on the order of about 150 nm such that the PI is can bind to the DNA of damaged or dead cells. If particles are too large, such intercalation event may not occur. If larger particles are desired for higher magnetic susceptibilities, as an example, carbon spacers may be used to increase the length/distance of propidium iodide (PI) from the surface of the particle to provide greater flexibility for intercalation of DNA. Particles smaller than approximately 30 nm may be problematic in that they are either not sufficiently magnetic and higher magnetic susceptibility core materials within a chosen magnetic energy field will have to be generated, or these small particles may contribute to nonspecific binding; that is, they may bind to viable cells as well as to dead and dying cells. If nonspecific binding relating to particle size is problematic, particle size may either be increased, or a blocking agent such as nonfat dried milk or serum albumin may be added to the labeling buffer solution to minimize nonspecific binding.

### Example 2

i. Removal of Damaged Sperm:

Fresh bull semen was obtained from a sample of four different bulls. Eight empty test tubes and a test tube with 5 mL of TRIS buffer were placed into a 35° C. water bath. After warming, 700 μL of warmed TRIS buffer was pipetted to four test tubes to be used as a control sample per bull, and 500 μL of TRIS buffer was pipetted to the other four test tubes to be used as the experimental sample per bull. From each bull, 40 million sperm cells were added to a control sample and an experimental sample. 200 μL of magnetic particles suspended in TRIS buffer, prepared as previously discussed, was added to each experimental tube. All samples were then incubated for 20 min., with shaking after 10 min. After incubation times had expired, tubes with cells and particles were placed on a Dexter magnetic stand where unbound particles and magnetically labeled cells migrated to the wall of the tube and were bound. Approximately 2 min. was allowed for the magnetic collection time, after which the nonmagnetic supernatant was aspirated using a transfer pipette and placed into a clean tube. All nonmagnetic fractions and control samples were analyzed for viability and total cell counts were measured via flow cytometry.

After removal of dead sperm, the percent viable is shown in TABLE 3:

TABLE 3

| | | % Viable After Magnetic |
|---|---|---|
| Sample ID | % Viable Control | Particles Diluted With TRIS |
| Bull A | 62.40 | 75.70 |
| Bull B | 70.57 | 76.47 |
| Bull C | 66.40 | 84.60 |
| Bull D | 77.50 | 79.40 |

From TABLE 3 it is observed that the percentage of viable sperm increased after removal of the dead sperm.

ii. Magnetically Labeled PI Saturation:

Sperm (3.8×10⁷ total) cryopreserved in a 0.5-ml straw were thawed. Aliquots containing about 5×10⁶ sperm were stained with PI-conjugated magnetic nanoparticles at a rate of 10, 50, 100, 200, 500, or 1000 μl. Aliquots were incubated at room temperature for 15 min, and subjected to the magnetic field from a magnetic test tube holder for separation. After separation, sperm were assayed using flow cytometry to identify the percentage of membrane intact (live) and membrane-damaged sperm. TABLE 4 illustrates that PI uptake reached saturation levels of about 100 μl of magnetic PI per 5×10⁶ sperm.

TABLE 4

| | Viable Sperm | Damaged Sperm |
|---|---|---|
| Particle | Negative Fraction Cell | Positive Fraction Cell Count |
| Volume (μL) | Count (percentage of Total) | (percentage of Total) |
| 10 | 37.5% | 62.5% |
| 50 | 45.2% | 54.8% |
| 100 | 62.1% | 37.9% |
| 200 | 61.4% | 38.6% |
| 500 | 62.7% | 37.3% |
| 1000 | 62.7% | 37.3% |

The foregoing description of the invention has been presented for purposes of illustration and description and is not intended to be exhaustive or to limit the invention to the precise form disclosed, and obviously many modifications and variations are possible in light of the above teaching. The embodiments were chosen and described in order to best explain the principles of the invention and its practical application to thereby enable others skilled in the art to best utilize the invention in various embodiments and with various modifications as are suited to the particular use contemplated. It is intended that the scope of the invention be defined by the claims appended hereto.

What is claimed is:

1. A method for separating sperm cells having damaged membranes from those having intact membranes comprising: attaching a membrane-impermeable, DNA-binding species to magnetic particles; mixing the resulting magnetic particles with a sample of sperm cells; and separating the sperm cells bound to magnetic particles by magnetic cell sorting.

2. The method of claim 1, wherein the membrane-impermeable, DNA-binding species comprises propidium iodide.

3. The method of claim 1, wherein the magnetic particles are coated by a silane compound having carboxyl groups.

4. The method of claim 3, wherein the silane compound comprises acidified 2-(carbomethoxy)ethyltrimethoxysilane.

5. The method of claim 4, further comprising the step of conjugating propidium iodide to the acidified 2-(carbomethoxy)ethyltrimethoxysilane.

6. The method of claim 4, wherein said step of conjugating propidium iodide is achieved using 1-ethyl-3-(3-dimethylaminopropyl)carbodiimide.

7. The method of claim 6, further comprising the step of quenching the conjugation reaction using a solution of glycine.

8. The method of claim 1, wherein said step of mixing the resulting magnetic particles with a sample of sperm further comprises the step of adding tris(hydroxymethyl)aminomethane to the mixture.

9. The method of claim 1, wherein said step of separating the sperm cells bound to magnetic particles by magnetic cell sorting comprises the steps of: applying a magnetic field to a container in which the sperm cells bound to magnetic particles are disposed; and removing the sperm cells which are not bound to the magnetic particles.

10. The method of claim 1, further comprising the step of selecting the sex of the sperm cells.

11. The method of claim 10, wherein sperm cells having damaged membranes are separated from those having intact membranes before said step of selecting the sex of the sperm cells.

12. The method of claim 10, wherein said step of selecting the sex of the sperm cells is performed using flow cytometry.

13. The method of claim 1, further comprising the steps of: detecting sperm having a chosen characteristic; and damaging the membranes of sperm cells not having the chosen characteristic.

14. The method of claim 13, wherein said step of damaging the membranes of sperms cells not having the chosen characteristic is achieved by applying a source of energy to the sperm cells not having the chosen characteristic.

15. The method of claim 14, wherein the source of energy comprises laser light.

16. The method of claim 14, wherein the source of energy comprises an electric charge.

17. The method of claim 13, wherein said step of detecting sperm having a chosen characteristic is performed using flow cytometry.

18. The method of claim 1, wherein the magnetic particles comprise magnetite.

19. The method of claim 1, wherein the magnetic particles comprise composites having the general structure MFe₂O₄ where M is chosen from Co, Ni, Cu, Zn, Mn, Cr, Ti, Ba, Mg, and Pt.

20. The method of claim 1, wherein the magnetic particles comprise nanoparticles.

21. A method for selecting sperm cells having a chosen characteristic comprising: detecting sperm cells having a chosen characteristic; damaging the membranes of sperm cells not having the chosen characteristic, forming thereby a mixture of sperm cells having damaged membranes and sperm having intact membranes; attaching a membrane-impermeable, DNA-binding species to magnetic particles; mixing the resulting magnetic particles with the mixture of sperm cells; and separating the sperm cells bound to magnetic particles by magnetic cell sorting; whereby the unseparated sperm have the chosen characteristic.

22. The method of claim 21, wherein said step of damaging the membranes of sperms cells not having the chosen characteristic is achieved by applying a source of energy to the sperm cells not having the chosen characteristic.

23. The method of claim 22, wherein the source of energy comprises laser light.

24. The method of claim 22, wherein the source of energy comprises an electric charge.

25. The method of claim 21, wherein said step of detecting sperm having a chosen characteristic is performed using flow cytometry.

26. The method of claim 21, wherein the membrane-impermeable, DNA-binding species comprises propidium iodide.

27. The method of claim 21, wherein the magnetic particles are coated by a silane compound having carboxyl groups.

28. The method of claim 27, wherein the silane compound comprises acidified 2-(carbomethoxy)ethyltrimethoxysilane.

29. The method of claim 28, further comprising the step of conjugating propidium iodide to the acidified 2-(carbomethoxy)ethyltrimethoxysilane.

30. The method of claim 28, wherein said step of conjugating propidium iodide is achieved using 1-ethyl-3-(3-dimethylaminopropyl)carbodiimide.

31. The method of claim 30, further comprising the step of quenching the conjugation reaction using a solution of glycine.

32. The method of claim 21, wherein said step of mixing the resulting magnetic particles with a sample of sperm cells further comprises the step of adding tris(hydroxymethyl)aminomethane to the mixture.

33. The method of claim 21, wherein said step of separating the sperm cells bound to magnetic particles by magnetic cell sorting comprises the steps of: applying a magnetic field to a container in which the sperm cells bound to magnetic particles are disposed; and removing the sperm cells which are not bound to the magnetic particles.

34. The method of claim 21, wherein the magnetic particles comprise magnetite.

35. The method of claim 21, wherein the magnetic particles comprise composites having the general structure MFe₂O₄, where M is chosen from Co, Ni, Cu, Zn, Mn, Cr, Ti, Ba, Mg, and Pt.

36. The method of claim 21, wherein the magnetic particles comprise nanoparticles.

37. The method of claim 21, wherein the chosen characteristic comprises the sex of the sperm cells.
